(19) United States
(12) Patent Application Publication
(10) Pub. No.: US 2026/0363552 A1
(43) Pub. Date: Nov. 19, 2026

(54) Bio-photonics based hand-held device

(71) Applicants: Stichting Het Nederlands Kanker Instituut - Antoni van Leeuwenhoek Ziekenhuis, Amsterdam (NL); University College Cork - National University of Ireland, Cork, Cork (IE)

(72) Inventors: Theodoor Jacques Marie Ruers, Zeist (NL); Ray Burke, Whitechurch, Co. Cork (IE)

(73) Assignees: Stichting Het Nederlands Kanker Instituut - Antoni van Leeuwenhoek Ziekenhuis, Amsterdam (NL); University College Cork - National University of Ireland, Cork, Cork (IE)

(21) Appl. No.: 19/142,982

(22) PCT Filed: Dec. 20, 2023
(86) PCT No.: PCT/EP2023/087095

§ 371 Date: Jun. 25, 2025

(30) Foreign Application Priority Data
Dec. 27, 2022 (NL) 2033846

(51) Int. Cl. A61B 5/00; A61B 10/02

(52) U.S. Cl. CPC A61B 5/0075; A61B 5/6848; A61B 10/0275; A61B 2560/0425; A61B 2560/0431; A61B 2560/045; A61B 2562/223; A61B 2562/228

(57) ABSTRACT

hand-held device (100) has a housing (200). The housing (200) comprises a first compartment (210) containing at least one first optical coupler (212) configured to transmit a light signal into an elongated optical component (300), at least one second optical coupler (213) configured to receive light signal from the elongated optical component (300), an optical sensor (410) optically connected to the at least one second optical coupler (213), and configured to detect the received light signal, a processor (420) configured to generate a parameter based on a signal generated by the optical sensor (410). The housing further comprises a second compartment (220) containing a placeholder (222) configured to receive at least a placement member (302) of the elongated optical component (300),

### FIELD OF THE INVENTION

The invention relates to a hand-held device for optical tissue examination.

### BACKGROUND OF THE INVENTION

For the detection and treatment of tumours in the human or animal body, it is important to correctly discern between healthy and pathological tissue. Biophotonic technologies are used for the analysis of biological materials. In healthcare, biophotonics devices have been developed to detect disease-specific biomarkers in tissues of human or animal bodies. That is the case of biphotonic probes, needles, or endoscopes that aid the physician to detect and differentiate tumours in, e.g., breast, lung, or gastrointestinal tract.

EP2358265B1 describes a biophotonic device having a needle with integrated optical fibres for tissue analysis. However, in general, there is a need for an improved biophotonic device.

### SUMMARY OF INVENTION

It would be advantageous to provide an improved biophotonic device.

Therefore, according to a first aspect of the invention, there is provided a hand-held device having a housing, the housing comprising:
a placeholder for receiving at least a placement member of an elongated optical component;
an attachment member to fix the elongated optical component to the housing (2), with the placement member disposed in the placeholder;
at least one first optical coupler configured to transmit a light signal into the elongated optical component;
at least one second optical coupler configured to receive a light signal from the elongated optical component; and
an optical sensor, optically connected to the at least one second optical coupler, and configured to detect the received light signal, and
a processor configured to generate a parameter based on a signal generated by the optical sensor.

The housing with the placeholder for the elongated optical component, as well as an optical sensor and processor built-in, allows to greatly simplify the operations of a biophotonic device. Any cable of optical fibres from the elongated optical component to an external console for detection and processing of optical signals, may become unnecessary. The device of the present disclosure may also improve the detection quality, because of the short optical track between the elongated optical component and the optical sensor.

The hand-held device may be provided to generate a parameter corresponding to disease-specific biomarkers in tissues of human or animal bodies. In this context, a hand-held device may be understood as a device having a housing that is compact and light enough to be held and manipulated with the hand. The placeholder and attachment member receive and secure at least part of the elongated optical component, that is used for simultaneous and bidirectional transfer of light between the examining site and the hand-held device. The elongated optical component may be of different types as described later below.

Providing an optical sensor in the hand-held device offers the advantage to reduce the distance that the light signal, received from the elongated optical component, travels before is processed, which improves the detection of the light signal and any deterioration of signal quality caused by long optical fibres may be avoided. Furthermore, a processor in the hand-held device offers the advantage to generate a parameter at the detection location of the received light signal, freeing the hand-held device from processing means outside the housing. In general, the hand-held device of the present disclosure may provide an autonomous integrated system that collects, detects and process optical information from examining tissues of the human or animal body. Such an autonomous integrated system may include an output signal that is perceptible by a human being, e.g., a visible or auditive signal, indicative of the parameter.

The elongated optical component may comprise optical fibres to conduct light signals between the examining site and the hand-held device. The first optical coupler may be configured to transmit the light signal from the housing into an optical fibre of the elongated optical component. The second optical coupler may be configured to receive the light signal from a second optical fibre of the elongated optical component. Optical fibres are an efficient way to transmit light signals through the elongated optical component in a controlled way.

The hand-held device may have a modular structure. Wherein the housing comprises a first compartment containing the at least one first optical coupler, the at least one second optical coupler, the optical sensor, and the processor, and a second compartment containing at least the placeholder and the attachment member, wherein the first compartment and the second compartment are detachable from each other. Detachable compartments of the housing facilitate the replacement of separate components of the hand-held device, which is convenient and cost effective if one of the compartments shall be replaced due to, for example, lifetime use. damage or disposability. Another advantage is that different embodiments of the second compartment, together with the elongated optical component, may be configured for examining different sites of the human or animal body, while using the same first compartment for detection and processing of the received signal. Thus, the present disclosure may also provide a versatile and compact device that may replace several other devices. In alternative implementations, the first compartment may be the housing and the second compartment may be part of the elongated optical component; for example, the second compartment may comprise the placement member of the elongated optical component which can be connected to a placeholder of the first compartment.

The housing may comprise a mechanism to mechanically advance at least a second elongated part of the elongated optical component over a first elongated part of the elongated optical component in a longitudinal direction of the elongated optical component, wherein the first elongated part may comprise the features of the elongated optical component described before, and wherein the mechanism is optionally spring-loaded. The mechanism provides versatility to the hand-held device, the elongated optical component may comprise multiple parts that may interact, depending on its purpose. Said second elongated part has the advantage to provide additional means to pierce tissues at the examining site for tissue collection, for example.

In another example of a modular design, the at least one first optical coupler, the at least one second optical coupler, the optical sensor and the processor are comprised in a first compartment of the housing, and wherein at least the attachment member and the mechanism are comprised in a second compartment of the housing, the first compartment being detachable from the second compartment, so that either compartment can be replaced. Similar to previous example, detachable compartments of the housing allow to replace separate components of the hand-held device, which is convenient and cost effective if one of the compartments shall be replaced due to, for example, lifetime use or damage. In this particular example, the first compartment and the second compartment may differ in cost and lifetime. The first compartment, containing optoelectronic components (i.e., optical sensor and processor), may be more expensive and durable than the second compartment, containing the attachment member and mechanism. In a further example, the second compartment may be a low-cost (semi-)disposable compartment.

The at least one first optical coupler and the at least one second optical coupler may align with the placeholder when the first compartment is attached to the second compartment. A mechanical alignment of the optical couplers with the placeholder facilitates the optical coupling, and improves the transmission quality of light signals, between the elongated optical component and the optical couplers.

The elongated optical component may be detachably attachable to the placeholder and to the attachment member, so that it can be replaced. A detachable elongated optical component allows for different types of elongated optical component to be used with the same hand-held device. The type might depend on the examining site and/or the application, for example biopsy, surgery, augmented imaging, etc, as exemplified later below. In a situation in which the examining site involves tissues inside the body, the elongated optical component is preferably sterile on the surfaces in contact with the body. A detachable elongated optical component has the advantage to be independent from the housing, facilitating either its sterilization or disposal.

The optical sensor may comprise at least one light wavelength detector. Specifically, the optical sensor may comprise at least one spectrometer configured to detect a spectrum of the received light signal and the processor may be configured to generate the parameter based on the detected spectrum. A spectrometer offers the advantage to detect light intensities in different wavelength ranges independently, which provides more information compared to a mere intensity detector and when the received light has a wide spectrum. The spectrometer may be adapted to detect a spectrum for, at least one out of the group consisting of, diffuse reflectance spectroscopy, (auto)fluorescence spectroscopy, differential path length spectroscopy, and Raman spectroscopy.

Alternatively, or additionally, as an implementation of a spectrometer, the optical sensor may comprise a set of discrete detector channels and a corresponding set of photodetectors, wherein each photodetector may be configured to detect light of a specific wavelength range in a detector channel of the set of discrete detector channels, and wherein the processor may be configured to compute the parameter based on signals generated by the set of photodetectors. The set of discrete detector channels may select light of specific spectrum, which facilitates the photodetectors to detect more specific wavelengths ranges. Photodetectors have the advantage to be highly sensitive to narrow wavelength ranges, which allow to detect wavelengths of particular interest. The discrete detector channels and photodetectors may be adapted to detect wavelengths from, at least one out of the group consisting of, reflectance spectrum, (auto)fluorescence spectrum, and Raman spectrum.

The housing may comprise an indicator configured to output information regarding the parameter. The indicator enhances the autonomy of the hand-held device by informing the user, in a condense manner, the properties of the tissues from the examining site, which provides a device that is more intuitive and easier to use. It may also remove the need for a separate console for visualization of the parameter.

The housing may comprise a wireless transmitter configured to wirelessly transmit the parameter to an external console. The wireless transmitter may be used to transmit the parameter for further processing, storage, and/or graphical displaying to the user. The wireless transmitter offers the advantage to transmit the parameter without the need of an additional cable, which enhances the ease to use the hand-held device.

The housing may comprise an optical port and a light guide configured to convey light signals received through the optical port from an external light source to the at least one first optical coupler. The optical port may convey optical signals with a wide spectrum or/and with specific wavelength rages, which offers the advantage to introduce more complex light signals for examination. Alternative or additionally, the housing may comprise a light source to generate a light signal, and a light guide configured to convey the light signal generated by the light source to the at least one first optical coupler. The light source integrated in the housing provides a short distance for the light signal to travel from its source to the examining site, which ensures the reliability of the light signal and enhances the autonomy of the hand-held device. The processor may be configured to control activation of the light source.

According to a second aspect of the invention, and in accordance with the advantages and effects described herein above, there is provided an elongated optical component comprising:
at least two optical fibres extending in a longitudinal direction through the elongated optical component, wherein the at least two optical fibres, are conductive to light signals, and
a placement member comprising at a proximal end of the elongated optical component, comprising at least one first optical coupler optically connected to at least one of the at least two optical fibres; and at least one second optical coupler optically connected to at least one of the at least two optical fibres; and
a connection element to allow fixation of the placement member,
   wherein a distal end of the first one of the at least two optical fibres and a distal end of the second one of the at least two optical fibres is optically open to an exterior of the elongated optical component.

The elongated optical component may transfer light simultaneously and bidirectionally between the examining site and the hand-held device. The transfer is performed by the optical fibres, which have the advantage to reliably conduct light to and from the examining site. The placement member fits in a placeholder and provides the at least one optical coupler with the correct alignment for the transmission of light signals. The connection element secures the placement member in the placeholder, ensuring a proper conduction of optical signals between the hand-held device and the elongated optical component. Because of the placement member and connection element of the elongated optical component may be removably connected to an external device, cleaning and/or replacement of the elongated optical component is facilitated.

The at least two optical fibres may be bundled as a bundle of optical fibres within the elongated optical component, wherein the at least two optical fibres branch away from each other at least at a distal end of the elongated optical component. Bundled optical fibres advantageously offer more mechanical flexibility, which facilitate their installation. They may also provide better signal to noise ratio than single fibres, and low shadowing effect, which is convenient when the conducted signals have low power. Furthermore, bundled optical fibres facilitate branching off the conducted optical signals, which is advantageous when the conducted signals are transferred to multiple detectors.

The elongated optical component may comprise a needle, which may have a rigid tubular body with small diameter. The needle may have the advantage to be able to puncture the body, facilitating the examination of deep tissues, e.g., internal organs. The needle may be configured as a biopsy needle, which advantageously collects a tissue sample from the examining site. This is particularly convenient when the elongated optical component is used to aid in the detection of potential pathological tissue.

A distal end of the elongated optical component may be hollow and comprise an insert, the insert may comprise at least one space for at least one of the optical fibres, wherein the at least one of the optical fibres extends through the space and is optically coupled to an exterior of the elongated optical component at the distal end of the elongated optical component. The insert offers the advantage to secure the optical fibres at the distal end of the elongated optical component. A further advantage is that the insert can maintain a proper distance and positioning of a distal end of the optical fibres, which may be important for proper processing and interpretation of the received optical signals by the processor.

The elongated optical component may comprise a biopsy needle. A distal portion of the elongated optical component may comprise a notch adapted to accommodate the tissue sample. Optionally, the elongated optical component may comprise two elongated parts: a first elongated part, being a needle and with the features described herein above and a second elongated part being a cutting canula slidably disposed around the first elongated optical component; and a handling member at a proximal end of the cutting canula configured to cooperate with an external mechanism to mechanically advance the cutting canula over the first elongated part. In this type, the notch may form a platform to accommodate the tissue sample. The cutting cannula has the advantage to cut the surroundings of the tissue sample, when it is advanced by the external mechanism and protects the tissue sample when the needle is withdrawn from the body.

According to a third aspect of the invention, and in accordance with the advantages and effects described herein above, there is provided a hand-held optical device, comprising:
a housing, the housing comprising:
   a placeholder for receiving at least a placement member of an elongated optical component;
   an attachment member to fix the elongated optical component to the housing, with the placement member disposed in the placeholder;
   at least one first optical coupler configured to transmit a light signal into the elongated optical component;
   at least one second optical coupler configured to receive a light signal from the elongated optical component; and
   an optical sensor, optically connected to the at least one second optical coupler, and configured to detect the received light signal, and
   a processor configured to generate a parameter based on a signal generated by the optical sensor; and
the elongated optical component comprising:
   an elongated optical component comprising at least two optical fibres extending in a longitudinal direction through the elongated optical component, wherein the at least two optical fibres are conductive to light signals, and
   a placement member comprising at a proximal end of the elongated optical component, comprising at least one first optical coupler optically connected to at least one of the at least two optical fibres; and at least one second optical coupler optically connected to at least one of the at least two optical fibres; and
   a connection element rigidly connected to at least the placement member to allow fixation the placement member,
      wherein the housing and the elongated optical component comprise alignment means so that the least one first optical coupler of the elongated optical component is aligned with said at least one optical coupler of the housing and the at least one second optical coupler of the elongated optical component is aligned with said at least one second optical coupler of the housing, and the placement member is removably fixable in the placeholder by the connection element and the attachment member. The elongated optical component may be disposable, which offers the advantage to not involve sterilization. This is particularly advantageous when the user has no access to equipment to sterilize components with small openings (for example, needles).

According to a fourth aspect of the invention, and in accordance with the advantages and effects described herein above, there is provided a method to assemble a biophotonics device. This aspect of the invention is particularly, but not exclusively, advantageous in that the method according to the present invention may be implemented using an apparatus according to the first and second aspects of the invention. The method comprising:
providing the hand-held device according to claim 1
providing the elongated optical component according to claim 14,
placing the placement member of the elongated optical component in the placeholder of the housing; and
connecting the attachment member of the hand-held device to the connection element of the elongated optical component.

The biophotonics device and method offers flexibility on its application to examine tissues of the human or animal body. The hand-held device may receive different types of the elongated optical component that are configured to examine different sites in the body or offer additional functionalities, which is convenient and cost effective.

The biophotonics device is provided with a structure that is easy to assemble. Placing the placement member of the elongated optical component in the placeholder of the of the housing is facilitated by the complementary shapes of the placeholder and placement member. This also provides alignment to the optical couplers for an effective transmission of light signals. Connecting the attachment member of the hand-held device to the connection element of the elongated optical component, secures the elongated optical component in the housing of the hand-held device to ensure a proper functioning.

According to a fourth aspect of the invention, and in accordance with the advantages and effects described herein above, there is provided a hand-held optical device. A hand-held optical device, comprising a housing, the housing comprising:
at least one transmitting fibre and at least one receiving fibre,
an optical sensor, optically connected to the at least one receiving fibre and configured to detect a light signal,
a light source optically connected to the at least one transmitting fibre and configured to generate a light signal, and
a processor configured to control activation of the light source and to generate a parameter based on a signal generated by the optical sensor,
   wherein a distal end of the transmitting fibre is configured to optically communicate with an outside of the housing and a distal end of the receiving fibre is configured to optically communicate with an outside of the housing.

The housing with the optical sensor and processor built-in, allows to greatly simplify the operations of a biophotonic device. Any cable of optical fibres from the housing to an external console for detection and processing of optical signals, may become unnecessary. The device of the present disclosure may also improve the detection quality, because of the short optical track between the examining site and the optical sensor. The hand-held optical device may be provided to generate a parameter corresponding to disease-specific biomarkers in tissues of human or animal bodies. In this context, a hand-held device may be understood as a device having a housing that is compact and light enough to be held and manipulated with the hand. In general, the hand-held optical probe of the present disclosure may provide an autonomous integrated system that collects, detects and process optical information from examining tissues of the human or animal body. Such an autonomous integrated system may include an output signal that is perceptible by a human being, e.g., a visible or auditive signal, indicative of the parameter. The hand-held device may comprise at least one battery to power the electronics. The battery may be replaced by any source of electric energy, such as a large capacitor and/or an energy harvesting component such as a solar panel. Alternatively, the device may be powered by an external power source.

### BRIEF DESCRIPTION OF DRAWINGS

In the following, aspects of the invention will be elucidated by means of examples, with reference to the drawings. The drawings are diagrammatic and may not be drawn to scale. Throughout the drawings, comparable items may be marked with the same reference numerals.

FIG. 1 shows a perspective view of a first example of a hand-held device according to the present disclosure.

FIG. 2A shows a first example of a modular configuration of the hand-held device and one first type of an elongated optical component.

FIG. 2B shows a second example of a modular configuration of the hand-held device and a second type of an elongated optical component.

FIG. 3A shows a partially exploded view of a first example configuration of the first compartment of the hand-held device with one light detector.

FIG. 3B shows a partially exploded view of a second example configuration of the first compartment of the hand-held device with two light detectors.

FIG. 4A shows an elongated optical component with two optical fibres.

FIG. 4B shows an elongated optical component with three optical fibres.

FIG. 5A shows a cross-section of the elongated optical component of FIG. 4B.

FIG. 5B shows a cross-section of an alternative elongated optical component with five fibres.

FIG. 5C shows a cross-section of an alternative elongated optical component with nine fibres.

FIG. 6A schematically shows a side view of a distal portion of an elongated optical component with a beveled tip.

FIG. 6B schematically shows a side view of a distal portion of an elongated optical component with a flat tip.

FIG. 6C schematically shows a side view of a distal portion of an elongated optical component with a concave tip.

FIG. 6D schematically shows a side view of a distal portion of an elongated optical component with a convex tip.

FIG. 7A shows a type of the elongated optical component arranged as a biopsy needle and with a moving canula in a first position.

FIG. 7B shows the elongated optical component of FIG. 5A and with the movable canula in a second position.

FIG. 8 shows a perspective view of another example of a hand-held device according to the present disclosure.

### DETAILED DESCRIPTION OF EMBODIMENTS

Certain exemplary embodiments will be described in greater detail, with reference to the accompanying drawings. The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail since they would obscure the description with unnecessary detail. Certain embodiments of the present disclosure can be used during medical procedures such as biopsy, surgery, etc. Yet, the present invention is suitable for any procedure that involves detecting and processing optical information from examining tissues of the human or animal body. Moreover, the devices disclosed herein may also advantageously be used in specimen analysis outside the human or animal body.

Although in the following detailed embodiments are presented with examples of a biopsy needle and a probe, the invention is not limited to this. For example, the presented devices may alternatively be integrated in a surgical device, such as a smart knife, or in a diagnostic tool, such as an ultrasound device or a thermometer. Optical sensor can be added to such a diagnostic tool so that optical sensing can be performed together with another modality such as ultrasound or temperature measurement.

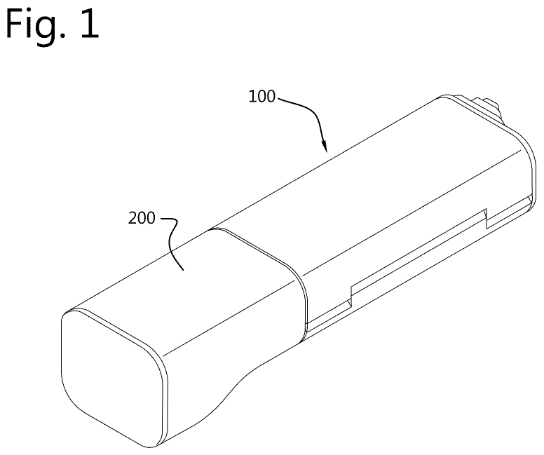

FIG. 1 shows a perspective view of a hand-held device according to the present disclosure. The hand-held device 100 comprises a housing 200. The housing 200 of the hand-held device 100 is compact and light enough to be held and manipulated with the hand. The housing 200 may have an ergonomic design, which facilitates the use of the hand-held device 100. Optional elements like buttons or sliders, may be easily manipulated with the same hand or may be manipulated with the other hand, depending on the user. For example, the housing may have a button to power the hand-held device and/or to start detecting. For example, the length of the housing is less than 200 mm, and both the width and height of the housing are less than 150 mm.

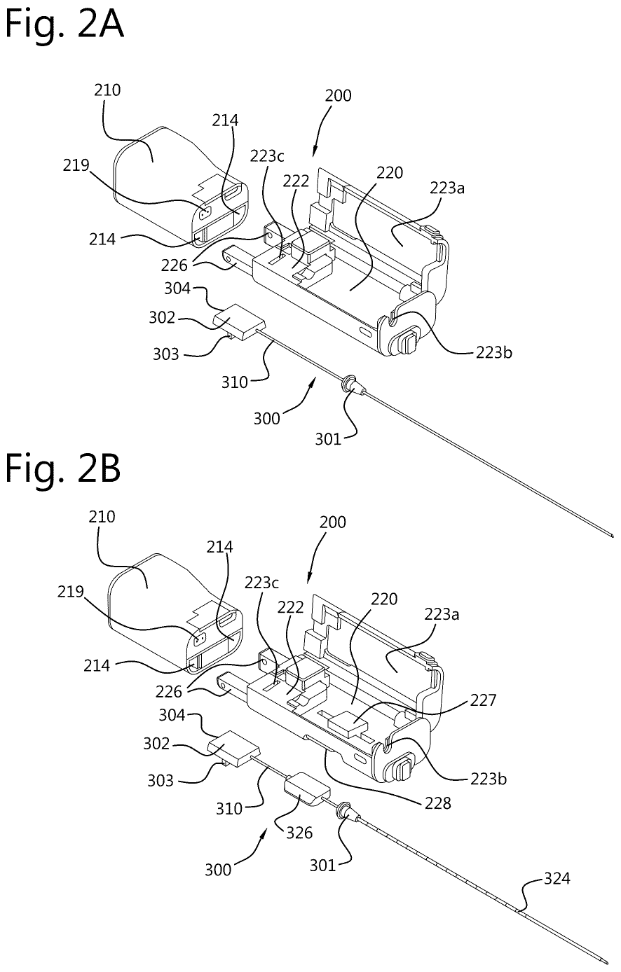

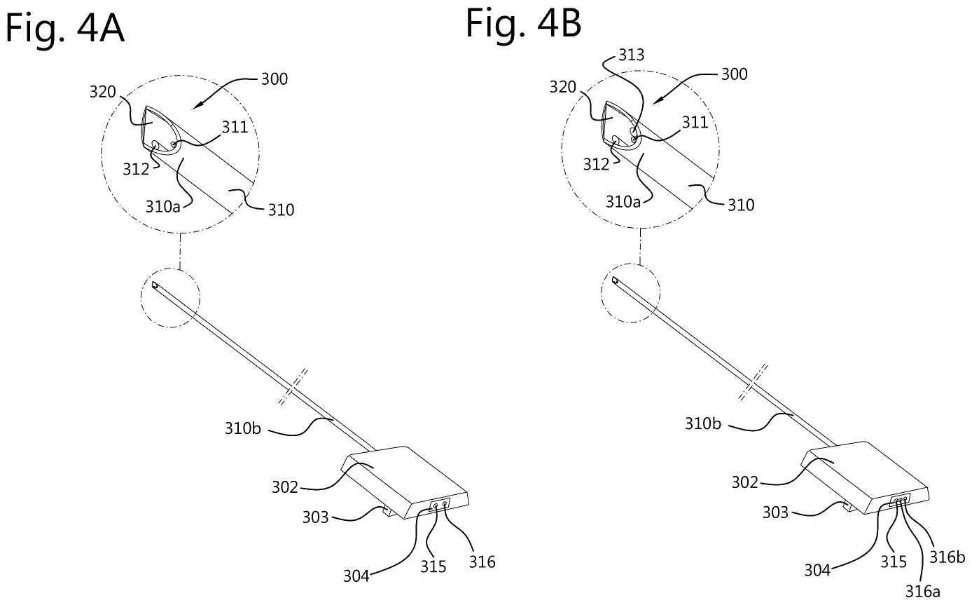

FIG. 2A shows a first example of a modular configuration of the hand-held device and one first type of an elongated optical component. The housing 200 comprises two detachable compartments, wherein the first compartment 210 is located at a proximal portion of the housing 200 and the second compartment 220 is located at a distal portion of the housing 200. The compartments 210 and 220 can mechanically connect to each other by any connection mechanism known in the art, such as a click mechanism or a magnetic connection. In the exemplary embodiment, the attachment arms 226 fit in spaces 214 to attach the second compartment to the first compartment. The second compartment 220 comprises in its interior a place holder 222 that receives a placement member 302 of an elongated optical component 300. An attachment member 223, comprised by a lid 223a and an opening 223b, removably fixes the elongated optical component 300 to the housing 200. The opening 223b allows at least part of the elongated optical component 300 to project outside the housing 200 and secures the elongated optical component 300 from a central portion via a ring 301. The ring 301 also guides the at least part of the of the elongated optical component 300 from the interior to the exterior of the housing 200. The lid 223a has a hinge that allows to easily open and close the housing 200 and facilitates positioning of the placement member 302 onto the place holder 222. Closing the lid 223a, attaches the elongated optical component 300 to housing 200. Additionally, or alternatively, the attachment member 223 may further comprise a socket 223c to receive and secure a connection element 303 of the elongated optical component 300. The connection element 303 may be rigidly connected to the placement member 302 and/or may protrude from the placement member 302. In some embodiments, the socket 223c may be absent and the placement element 302 may also be the connection element 303 by having a complementary shape to that of the placeholder 222 and the lid 223a. In other embodiments, the lid 223a of the housing is not openable and the opening 223b may be a slit to allow the placement member 302 to be inserted in the housing 200. It will be understood by the skilled person that the fixation of the placement member 302 to the housing may be modified or substituted by any fixation or alignment mechanism to keep the placement member accurately at its place. In another implementation, the elongated optical component 300 may be part of, or be permanently attached to, the second compartment 220. The placement member 302 may be integrally formed with the place holder 222 and the second compartment may not have the openable lid 223a. In this implementation the second compartment 220, together with the elongated optical component 300 may be disposable. A first part of an optical interconnector 219 in the first compartment 210 may be configured to receive a second part 304 of the placement member 302 (see FIG. 4), which is positioned in the placeholder 222. The optical interconnector may be a known connector, for example, a multi-fibre push-on (MPO) connector.

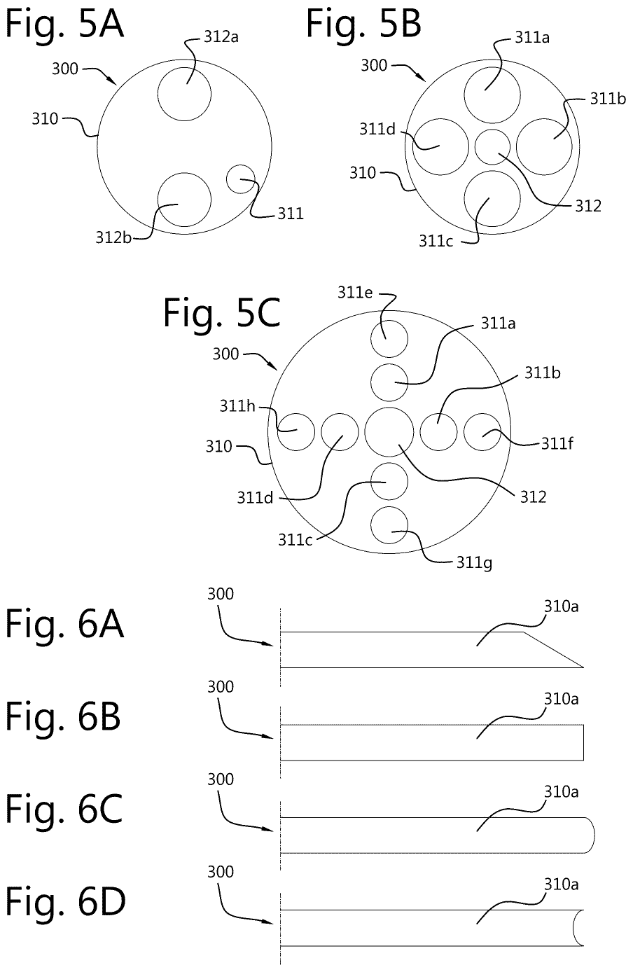

FIG. 2B shows a second example of a modular configuration of the hand-held device and a second type of an elongated optical component. The second compartment 220 may further comprise an optional mechanism 227 to mechanically interact with at least one part 326 of the elongated optical component 300, which provides additional functionalities (see below with reference to FIGS. 5A and 5B). The mechanism may be automatically or semi-automatically trigged by the user by at least one button 228 on the housing 200. In a preferred embodiment, the mechanism is spring loaded that is lock/unlocked by a first button and triggered by a second button, on the housing.

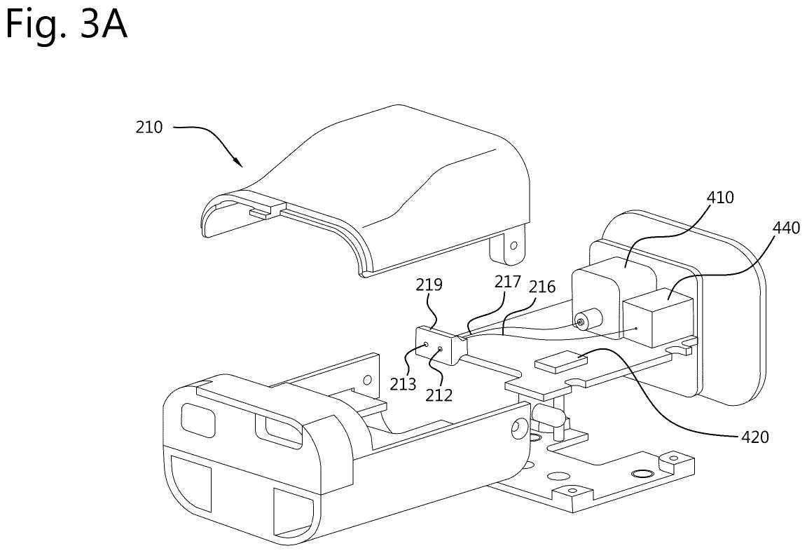

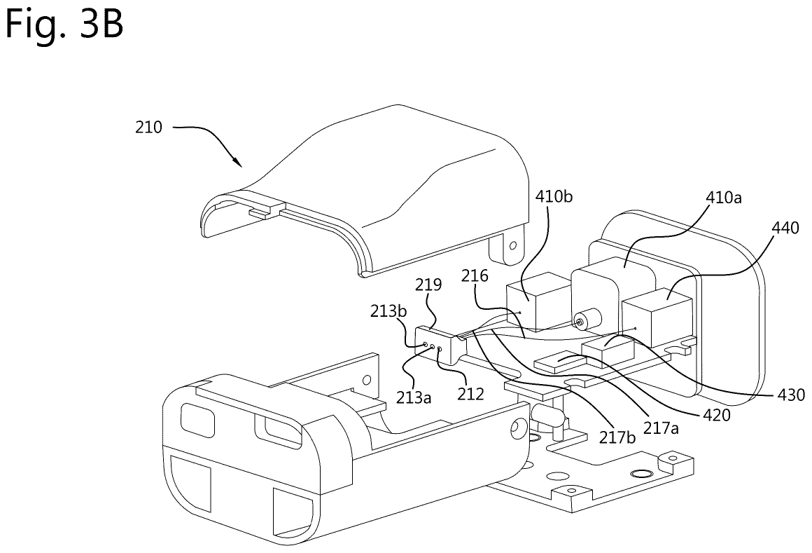

FIG. 3A shows a partially exploded view of a first example configuration of the first compartment of the hand-held device with one light detector. A first optical coupler 212 and a second optical coupler 213 are contained in the first compartment 210 and arranged in the first part of the optical interconnector 219. The mechanical alignment of the optical interconnector (219 and 304) may have a maximum tolerance of about 10 microns. In this manner light signals travel effectively between the elongated optical component 300 and optoelectronic components contained in the first compartment 210, more specifically between the optical couplers 212 and 213 and two couplers (315 and 316) in the placement member 302 (described later below with reference to FIG. 4).

The abovementioned couplers may be butt couplers, which advantageously provide a minimal optical path signal loss. The maximum optical path signal loss of the optical couplers may have a range between about 0.5 dB to 1.2 dB, which provides an effective transmission of light for an accurate detection. The first part of the optical interconnector 219 comprises at least one optical guide for each optical coupler. A first optical guide 216 is connected to or integrally formed with the first optical coupler 212. Similarly, a second optical guide 217 is connected to or integrally formed with the second optical coupler 213. A hand-held device with detachable housing compartments is convenient and cost efficient if one of the compartments shall be replaced due to, for example, lifetime use or damage. It is noted that the detachable compartments are optional. Alternatively, the compartments may be permanently fixed to each other and/or their respective elements may be fully integrated a single housing. Thus, the hand-held device of the present disclosure may provide an autonomous integrated system that collects, detect and process optical information from examining tissues of the human or animal body without the need of detachable compartments.

In the first example of the first compartment (FIG. 3A), the first optical coupler 212 transmits light signal from an internal light source 440 via the first optical guide 216 into the elongated optical component 300 through the optical interconnector. Optionally, the first optical coupler 212 transmits light signal into the elongated optical component 300 from an optical port optically connected to an external light source. The light source may comprise multiple internal and/or external light sources, as well as multiple optical ports are plausible to transmit light to multiple first optical couplers. Alternatively, multiple internal and/or external light sources may be optically switched, split, or combined to transmit light to one or more first optical couplers. The light source, either internal or external may be one or combinations of a broadband light source (for example, tungsten-halogen or mercury lamp) and one or more narrow band light source (for example, laser, light-emitting diode or filtered broadband light).

FIG. 3A shows an optical sensor 410 optically connected to the second optical coupler 213 via the second optical guide 217. The second optical coupler 213 receives light from the elongated optical component 300. The optical sensor 410 may be a photodiode or a broadband light detector. The optical sensor 410 may be a light detector to detect a spectrum of the received light signal, for example a spectrometer that generates electrical signals depending on the detected spectrum. The spectrometer may be a super dispersion micro spectrometer configured to detect a spectrum of light with wavelength bands suitable for, e.g., one or more of diffuse reflectance spectroscopy (DRS), auto-fluorescence spectroscopy (AFS), differential path length spectroscopy, and Raman spectroscopy (including the following sub classifications or variations SERS, SORS and INVERSE SORS). In a preferred embodiment, optical sensor 410 is a spectrometer configured to detect a DRS spectrum in a spectral band between about 300 nm to 1700 nm and preferably between about 300 nm to 940 nm. The sensitivity of the DRS measurement may depend on the light absorbance and scattering of the target biomarker as well as the specifications of the spectrometer and the light source. A processor 420, connected to the optical sensor 410, is configured to process the signals from the optical sensor 410, to generate a parameter based on the electrical signal generated by the optical sensor 410. For example, the parameters may be calculated by the processor using known algorithms for one or more of diffuse reflectance spectroscopy (DRS), auto-fluorescence spectroscopy (AFS), differential path length spectroscopy, and Raman spectroscopy (including the following sub classifications or variations SERS, SORS and INVERSE SORS).

In a general operation, the hand-held device 100 transmits a light signal from the light source 440 into the elongated optical component 300 via the optical coupler 212. In this context, a light signal intends to denote an electromagnetic signal which may comprise signals of a spectrum including but not limited to visible light and/or infrared light. The elongated optical component 300 conducts said light signal to biological tissues of the examining site. The tissues absorb or back-scatter the light signal and generate a reflectance light signal that is collected by the elongated optical component 300 and received by the hand-held device 100. The reflectance light signal has a spectrum containing information about the optical properties and structure of the biological tissues. The processor 420 in the hand-held device transforms the detected spectrum into at least one physiological parameter that is indicative of the tissue state (e.g., pathological, or not pathological). The processor may be configured to perform algorithmic calculations based on the optical signals to generate the at least one physiological parameter. Alternatively, the processor may be configured to perform calculations based on artificial intelligence, for instance, custom intelligent application specific algorithms. The hand-held device offers the advantage to detect and process the light reflected by the tissues at a short distance from the examining site, that is of about 200 mm, minimising the distance that the received light signals travel and enhancing the accuracy and reliability of the physiological parameter. Optionally, the housing comprises visual or audio indicators to give information to the user regarding the physiological parameter.

FIG. 3B shows a partially exploded view of a second example configuration of the first compartment of the hand-held device with two light detectors. The housing 200 comprises two second optical couplers 213a and 213b that receive light from the elongated optical component 300. Consequently, two second optical guides 217a and 217b are respectively connected to or integrally formed with the second optical couplers 213a and 213b. The second optical guides 217a and 217b are respectively optically connected to optical sensors 410a and 410b. The first optical sensor 410a may be a spectrometer and have the same characteristics of that of the optical sensor 410 in the configuration shown in FIG. 3A. The second optical sensor 410b may be e.g., a photodiode or a broadband light detector. The second optical sensor 410b may comprise a second micro spectrometer and may be configured to detect a different spectrum to that of the first optical sensor 410a, wherein the second spectrometer 410b may be configured for, at least one out of the group consisting of, diffuse reflectance spectroscopy (DRS), autofluorescence spectroscopy (AFS), differential path length spectroscopy, and Raman spectroscopy (including the following sub classifications or variations SERS, SORS and INVERSE SORS). Alternatively, the second optical sensor 410b may comprise a set of discrete detector channels and a set of photodetectors that detect light of a specific wavelength range and generate electrical signals depending on the detected light. Each detector channel may narrow down the band of the received light, subsequently, each detector channel is connected to a photodetector that detects light of a different wavelength range. The set of photodetectors may be a set of Silicon Photo Multipliers (SiPM) that offer the advantage of being detectors with a small size (<100 microns), high sensitivity to light (about to 40 pico-Watts of optical power), and with a high gain (>10⁶). The number and the band of the detector channels and photodetectors depends on the wavelengths of the biomarkers of interest. For example, three detector channels and three photodetectors could be used to detect the light in three wavelength ranges. The processor 420, connected to the optical sensors 410a and 410b, generate at least one parameter based on the electrical signals generated by the optical sensors 410a and 410b, wherein the at least one parameter may be a physiological parameter indicative of the tissue state (e.g., detection of a certain tissue type or biomarker, concentration of a particular substance, indication if a certain threshold concentration is exceeded, etc.). The parameter may be computed, for example, using algorithmic calculations on the detected signals. Alternatively, the parameter may be computed using a custom intelligent application specific algorithm.

In an alternative implementation, the same optical sensor (410a or 410b) is used for one or more optical couplers (213a, 213b, 213c, 213d . . . ). For example, an optical switch may be provided so that either one of the optical couplers is connected to the optical sensor at any given time. The switch may be controlled by the processor 420, for example, to select active optical couplers and their corresponding optical fibres of the elongated optical component 300, according to the desired type of measurement.

In preferred embodiment of the second example of the first compartment (FIG. 3B), the hand-held device is configured to perform DRS and AFS spectroscopy of the examining site. Measuring both DRS and AFS offer the advantage to measure different types of optical properties of the examining tissues, providing a more accurate and reliable identification of disease-specific biomarkers. The light source 440 provides light with a broad band and with multiple spectral bands in a switched and timed fashion. The processor 420 coordinates the light source 440 to provide broadband light and each of the multiple spectral bands for certain period in a predetermined and repeated sequence. The number and wavelength range of the multispectral bands corresponds to the number and wavelength range of the discrete detector channels and photodetectors. The processor 420 synchronizes the optical sensors 410a and 410b with the light source 440 such that on periods providing broadband light, the received light signals are detected by the first optical sensor 410a. Respectively, on the period of each multispectral band, the received light signal is detected by the second optical sensor 410b and specifically by the corresponding detector channel and photodetector. In this example the processor 420 is configured to process and generate at least one parameter based on the electrical signals of both optical sensors 410a and 410b. This processing may involve a custom intelligent application specific algorithm.

The optical components of any of the abovementioned examples may be interconnected and powered with an embedded electronic system, which provides a compact and effective integration suitable for the housing 200 of the hand-held device 100. The hand-held device can be adapted to include multiple first and second optical couplers with multiple optical sensors and light sources, for example, the couplers may be switched to the same optical sensor or light source. Moreover, the processor 420 may be a known processor capable to control the available optoelectronic components.

In some embodiments, the hand-held device comprises a wireless transmitter 430 to transmit the physiological parameter or other parameters possibly generated by the sensor to an external console or display. The wireless transmitter 430 has the advantage to transmit more detailed data detected in the hand-held device, which may provide the user with graphical visualization of one or more physiological parameters. Moreover, the hand-held device may comprise at least one battery to power the electronics. For example, in a two-component housing, the battery may be included in either one of the components. An electric connection between the housings may be provided, so that the other compartment can be powered by the compartment with the battery. However, it is also possible to include a separate battery on each compartment. In certain implementations the second compartment may be fully mechanical and does not include electronics, so that it does not need a battery. The battery allows the device to operate autonomously. The battery may be replaced by any source of electric energy, such as a large capacitor and/or an energy harvesting component such as a solar panel. Alternatively, the device may be powered by an external power source.

FIG. 4A shows an elongated optical component with two optical fibres. The elongated optical component 300 has a tubular body 310. Two optical fibres 311 and 312 extend in a longitudinal direction through the elongated optical component 300 and are optically connected to a first 315 and a second 316 optical couplers of the placement member 302 at a proximal end 310b of the elongated optical component 300. The optical fibres 311 and 312 are (at least during operation) optically open to an exterior of the elongated optical component at both proximal and distal ends. The optical fibres 311 and 312 may be integrated in the side wall, or glued to an interior, of the tubular body 310.

The elongated optical component 300 has the purpose to transfer light simultaneously and bidirectionally between the examining site and the optical couplers 315 and 316. Thus, the optical fibres 311 and 312 are conductive to light signals. In the example of FIG. 4A, the first fibre 311 is a transmitting fibre that conducts light from the first optical coupler 315 to the distal end 310a of the elongated optical component 300, while the second fibre 312 is a receiving fibre that conducts light from the distal end 310a to the second optical coupler 316. The optical couplers (315 and 316) are comprised in the second part of the optical interconnector 304 that is integrated in the placement member 302. In the illustrated example, the distal end 310a of the tubular body 310 is hollow, and the optical fibres 311 and 312 are arranged in an insert 320. The insert 320 comprises one space for each of the optical fibres and secures their distal end in the tubular body. The insert 320 may be configured to provide an optimal distance between the distal end of the optical fibres for an accurate measurement. For example, the elongated optical component 300 of FIG. 4A may be configured to conduct light signals to be used for DRS. The first fibre 311 conducts light signals into the tissues of the examining site and the second fibre 312 conducts reflected light by the tissues. The insert 320 secures the optical fibres 311 and 312 at a distal end 310 a with a distance between about 0.7 and 1 mm. In certain embodiments, the fibre distances between transmitting and collecting fibres may vary meaning that the distal end point of different collecting fibres may have different distance from the distal end point of the transmitting fibre, as illustrated by means of example in FIG. 5A. The distal end points of the collecting fibres may also be spatially distributed around the transmitting fibre, as illustrated by means of example in FIGS. 5B and 5C. For example, different distances can be measured at once using multiple collecting fibres.

FIG. 4B shows an elongated optical component with three optical fibres. In all illustrated examples, the elongated optical component may be rigid or semi-flexible (e.g., comprise a metal), forming a straight stick or tube, as opposed to a flexible cable. That is, there is no cable connection between the housing and the elongated optical component. Rather, the proximal end of the elongated optical component may be directly fixed to the housing and may be partially inserted into the housing. The third optical fibre may be used as a second receiving fibre 312b to detect a second type of reflected light from the tissues. Alternatively, the third optical fibre may be used to conduct a second type of light signals into the tissues. In a preferred embodiment, the elongated optical component 300 of FIG. 4B is configured to conduct light signals to be used for DRS and AFS, in which the fibres 311 and 312a have the same configuration as in the example of FIG. 4A suitable for DRS, and the third fibre 312b is configured to conduct light signals from the tissues for AFS to an additional second optical coupler 316b. In this example, the insert 320 secures the optical fibre 312b adjacent to fibre 311 at a distal end 310a of the tubular body 310 as the autofluorescence light signals have low intensity and involve an inelastic process. The optical fibres 311 and 312 may have a diameter between about 100 micrometres and 400 micrometres.

The optical fibres may be bundled as a bundle of optical fibres within the elongated optical component 300, wherein the insert 320 and the placement member 302 separate the optical fibres from each other at the distal end 310a and proximal end, respectively, of the elongated optical component 300. In a preferred embodiment, the tubular body 310 of the elongated optical component 300 has the diameter of a needle to be able to be inserted in the body. In the examples of FIGS. 4A and 4B, the insert 320 and the distal end 310a have a beveled tip, which provides the elongated optical component 300 with puncture properties to reach deep tissues of the human or animal body. The bevel angle may have a range from about 20 deg to 45 deg, which allows for an adequate arrangement and polishing of the optical fibres. Furthermore, given that the examining site typically has a high refractive index, i.e., it comprises biological tissues and water, the bevel angle provides an effective light transfer between the elongated optical component 300 and the examining. In other implementations, the tubular body 310 may have a larger diameter with a blunt tip. As a result, the elongated optical component 300 may be used as a probe that can be put on a tissue, just touching the outside of the tissue without puncturing.

The insert 320 may be made from machined or moulded metallic and/or plastic, in a preferred embodiment, the insert is made of micro moulded LCP polymer. It will be understood by the skilled person, that multiple fibres are covered by the scope of the present disclosure and that the diameter of the tubular body 310 and the arrangement of the insert can be adapted accordingly. The insert 320 and the distal end 310a are beveled and sharpened to allow the elongated optical component 300 to pierce the tissues and to allow the optical fibres to be integrated to the tip and polished in situ. The insert 320 and the distal end 310a may have multiple geometries or cutting geometries depending on the distance between fibres at the tip, such to optimize the fibres' placement and the cutting properties. The distal end may have multiple complex surfaces and planes on the needle tip to maximise its mechanical and sensor performance.

FIG. 5A shows a cross-section of the elongated optical component of FIG. 4B. FIG. 5B shows a cross-section of an alternative elongated optical component with five optical fibres suitable for measurement of AFS and Raman spectroscopy, in which four transmitting fibres 311a-d surround a receiving fibre 312. FIG. 5C shows a cross-section of an alternative elongated optical component with a larger diameter and nine optical fibres suitable for measurement of DRS and SORS, in which eight transmitting fibres 311a-h surround a receiving fibre 312. Fibres 311a-d send light signals to the tissues for a first stage of SORS and fibres 311e-h send light signals to the tissues for DRS and a second stage of SORS. The cross sections shown in FIGS. 5A-5C show the cross section at the distal end of the elongated optical component. As described above, in the interior of the elongated optical component the fibres may be bundled.

FIG. 6A-D schematically show a side view of different configurations of the distal portion of the elongated optical component. A beveled tip (FIG. 6A), as shown before, offers the advantage of facilitating puncturing of the body to reach deep tissues. A flat tip (FIG. 6B) has the advantage to be easy to manufacture and to reduce internal reflection at the tip of the needle. A concave or a convex tip (FIG. 6C-D) may be appropriate depending on the intended optical interaction between the transmitted light and the tissues as well as on the measured biomarkers.

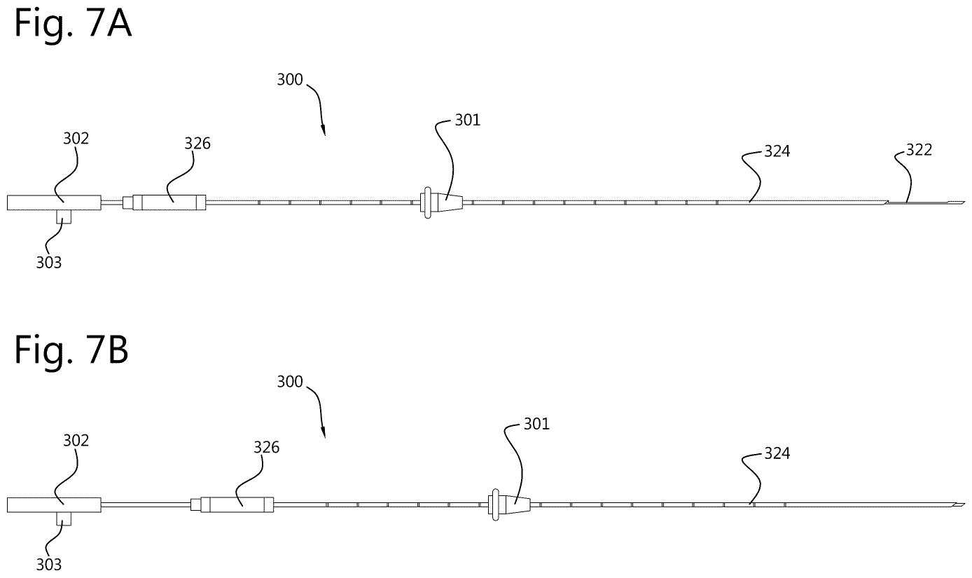

FIGS. 7A and 7B show side views of a type of the elongated optical component arranged as a biopsy needle and with a moving canula in a first position. The tubular body 310 may have a notch 322 or cavity at a distal portion 310a. In the exemplary embodiment, the notch 322 is located adjacent and proximally to the insert 320 and forms a platform adapted to accommodate a tissue sample. The radial position of the notch 322 may be opposite with respect to the bevel at the distal end 310a of the tubular body 310 (as shown in FIGS. 7A and 7B) or may have a different orientation. The notch 322 may have a length between about 10 and 30 mm, which accommodates a sample suitable for laboratory analysis. In a preferred embodiment the tubular body 310 has a length between about 15 and 25 mm, which allows the elongated optical component 300 to reach internal organs in the body. The hand-held device 100 used with an elongated optical component 300 configured as a biopsy needle offers the advantage to aid the user to select the most optimal place for a biopsy and collect potentially pathological tissue there for further analysis in a laboratory, which improves the accuracy of the biopsy collection and reduces the hazard to the patient of collecting multiple biopsies.

The elongated optical component 300 of FIGS. 7A and 7B further comprises a cutting cannula 324 slidably disposed around the tubular body 310. FIG. 7A shows a first position in which the notch 322 is exposed. FIG. 7B shows a second position in which the notch 322 is covered by a distal portion of the cannula 324. The cannula 324 may be configured to move over the tubular body 310 from the second position to the first position, and to advance back from the first position to the second position, under control of the mechanics of the second component of the housing. The handling member 326 is attached to a proximal end of the cannula 324 and is configured to cooperate with an external mechanism to mechanically advance and/or retract the canula 324 over the tubular body 310. The canula 324 may be advanced to cut a sliver of tissue sample and further secure it in the elongated optical component 300 to be withdrawn from the body. Although not visible in the perspective view of FIGS. 7A and 7B, the elongated optical component 300 of FIGS. 7A and 7B also comprise optical fibres as described with reference to the other figures.

The elongated optical component 300 may be reusable or disposable but preferably disposable. The simple and convenient design of any of abovementioned types facilitate a structure that can inexpensively be manufactured without affecting performance, offering a cost effective elongated optical component that can be disposed and thus does not have the need to be sterilized.

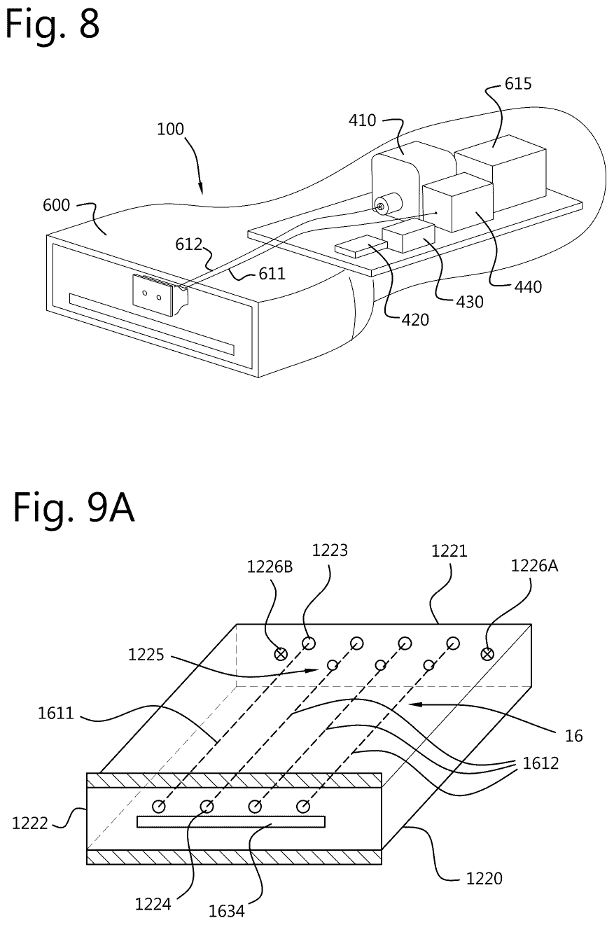

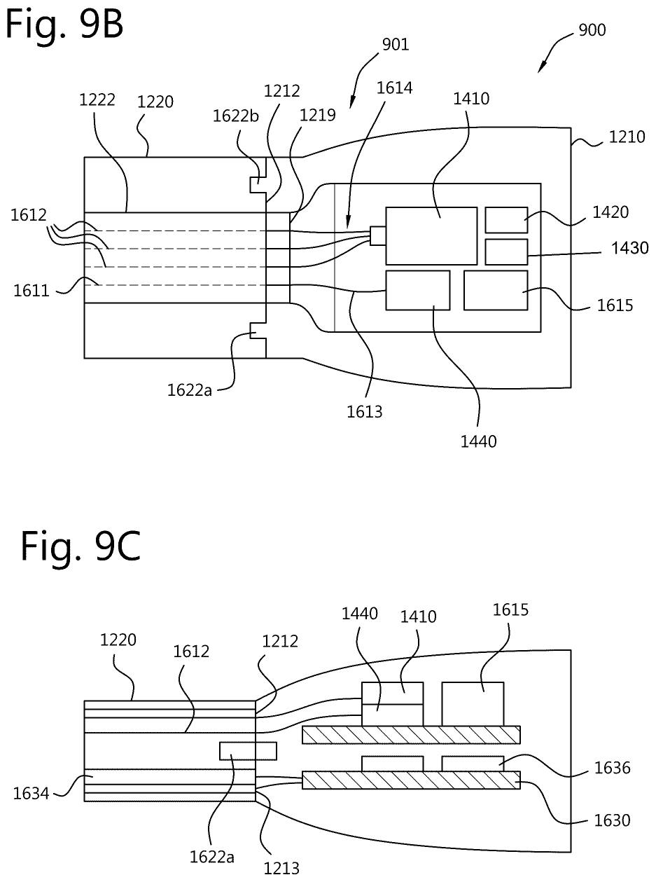

FIG. 8 shows a perspective view of another example of a hand-held device according to the present disclosure. The hand-held device 100 comprises a housing 600. The housing 600 is compact and light enough to be held and manipulated with one hand and may have an ergonomic design, which facilitates the use of the hand-held device 100. Optional elements like buttons or sliders, may be easily manipulated with the same hand or may be manipulated with the other hand, depending on the user. For example, the housing may have a button to power the hand-held device and/or to start detecting. The housing 600 comprises at least one transmitting fibre 611 and at least one receiving fibre 612. The at least one transmitting fibre 611 transmits light signals from an internal light source 440, at its proximal end, to the examining site, wherein a distal end of the at least one transmitting fibre 611 optically communicates with an outside of the housing 600. Optionally, the at least one transmitting fibre 611 transmits light signal from an optical port optically connected to an external light source. The light source may comprise multiple internal and/or external light sources, as well as multiple optical ports are plausible to transmit light to multiple transmitting fibres. Alternatively, multiple internal and/or external light sources may be optically switched, split, or combined to transmit light to one or more transmitting fibres. The light source, either internal or external may be one or combinations of a broadband light source (for example, tungsten-halogen or mercury lamp) and one or more narrow band light source (for example, laser, light-emitting diode or filtered broadband light).

The first optical fibre 611 conducts light signal to biological tissues of the examining site. The tissues absorb, reflect, or back-scatter the light signal and generate a reflectance light signal that is collected at a distal end of the at least one receiving fibre 612, where the receiving fibre 612 optically communicates with the outside of the housing 600. The reflectance light signal has a spectrum containing information about the optical properties and structure of the biological tissues. An optical sensor 410, optically connected to a proximal end of the at least one receiving fibre 612, may be a photodiode or a broadband light detector. The optical sensor 410 may be a light detector to detect a spectrum of the received light signal, for example a spectrometer that generates electrical signals depending on the detected spectrum. The spectrometer may be a super dispersion micro spectrometer and may be configured to detect a spectrum of light with wavelength bands suitable for, e.g., one or more of diffuse reflectance spectroscopy (DRS), auto-fluorescence spectroscopy (AFS), differential path length spectroscopy, and Raman spectroscopy (including the following sub classifications or variations SERS, SORS and INVERSE SORS). In a preferred embodiment, optical sensor 410 is a spectrometer configured to detect a DRS spectrum in a spectral band between about 300 nm to 1700 nm and preferably between about 300 nm to 940 nm. The sensitivity of the DRS measurement may depend on the light absorbance and scattering of the target biomarker as well as the specifications of the spectrometer and the light source.

A processor 420, connected to the optical sensor 410, is configured to control activation of the light source 440 and to generate a parameter based on the electrical signal generated by the optical sensor 410, wherein the parameter may be at least one physiological parameter that is indicative of the tissue state (e.g., pathological, or not pathological). For example, the parameter may be calculated by the processor using known algorithms for one or more of diffuse reflectance spectroscopy (DRS), auto-fluorescence spectroscopy (AFS), differential path length spectroscopy, and Raman spectroscopy (including the following sub classifications or variations SERS, SORS and INVERSE SORS). The processor 420 may be configured to perform algorithmic calculations and/or Custom Intelligent application specific algorithms, when processing the optical signals to generate the at least one physiological parameter. The hand-held device offers the advantage to detect and process the light reflected by the tissues at a short distance from the examining site, that is of about 200 mm, minimising the distance that the received light signals travel and enhancing the accuracy and reliability of the physiological parameter. The hand-held device 100 may comprise a wireless transmitter 430 to transmit the parameter or other parameters possibly generated by the sensor to an external console or display. The hand-held device may be combined with hardware for other type of optical measurements, for example, an ultrasound transducer, which improves the specificity of the hand-held device to analyse tissues the examining site.

To improve its autonomy, the hand-held optical device 100 may include an output signal that is perceptible by a human being, e.g., a visible or auditive signal, indicative of the parameter. The hand-held device may further comprise at least one battery 615 to power the electronics. The battery may be replaced by any source of electric energy, such as a large capacitor and/or an energy harvesting component such as a solar panel. Alternatively, the device may be powered by an external power source.

According to an aspect, a hand-held optical device is provided, comprising a housing, wherein the housing comprising:
a first compartment comprising:
at least one transmitting optical fibre extending inside the first compartment from a distal side of the first compartment,
and at least one receiving optical fibre extending inside the first compartment (1210) from the distal side of the first compartment,
an optical sensor, optically connected to the at least one receiving optical fibre and configured to detect a light signal,
a light source optically connected to the at least one transmitting optical fibre and configured to generate a light signal; and
a processor configured to control activation of the light source and to generate a parameter based on a signal generated by the optical sensor, and
connection means;
the housing further comprising a second compartment comprising:
at least one transmitting optical fibre extending from a proximal side of the second compartment to a distal side of the second compartment,
at least one receiving optical fibre extending from the proximal side of the second compartment to a distal side of the second compartment, and
connection means configured to cooperate with the connection means of the first compartment to detachably attach the first compartment to the second compartment,
wherein a distal end of the transmitting optical fibre of the second compartment is configured to optically communicate with an outside of the housing and a distal end of the receiving optical fibre of the second compartment is configured to optically communicate with an outside of the housing, and
wherein the connection means of the first compartment and the distal end of the transmitting optical fibre of the first compartment and the distal end of the receiving optical fibre of the first compartment are aligned with the corresponding connection means of the second compartment and the corresponding proximal end of the transmitting optical fibre of the second compartment and the corresponding proximal end of the receiving optical fibre of the second compartment so that the optical fibres of the first compartment are in optical connection with the optical fibres of the second compartment when the first compartment is attached to the second compartment by the connection means of both compartments.

This provides a hand-held optical measurement tool of which the most distal part (the part contacting the measured sample) may be replaced. This provides for cost efficient operation because the first compartment with the relatively expensive optoelectronic components may be used during their entire lifetime while replacing the second compartment whenever need be, for example.

In certain embodiments, the housing of both the first compartment and the second compartment is rigid.

In certain embodiments, the optical fibres in the second compartment may extend linearly through the second compartment.

The first compartment may comprise at least one electrical connection at the distal side of the first compartment; and the second compartment may comprise at least one electrical connection at the proximal side of the second compartment. The at least one electrical connection at the distal side of the first compartment may be aligned with the at least one electrical connection at the proximal side of the second compartment to allow transmission of electrical signals when the first compartment is attached to the second compartment by the connection means of both compartments. For example, the electrical connections of both compartments may be located at corresponding distances from the connection means. This makes it possible to provide electrical power supply and/or electrical control signals, data signals, or any other electrical signals, from the first compartment to the second compartment and vice versa. For example, the exchange of control signals and measurement signals for an ultrasound transducer may be exchanged between the compartments via the electric connections.

FIGS. 9A through 9C illustrate another hand-held optical device 900.

FIG. 9A schematically shows a perspective view of a compartment that forms a second compartment 1220 of the hand-held optical device 900. The second compartment 1220 comprises a plurality of optical fibres 1611, 1612 extending therethrough from a proximal side 1221 to a distal side 1222 of the second compartment 1220. At the distal side 1222 the optical fibres may be configured to exchange optical signals with an environment of the hand-held optical device 900, in particular with a sample to be measured, for example through respective holes 1224 in the housing of the second compartment 1220. Similarly at the proximal side 1221 the optical fibres may be configured to exchange optical signals with the first compartment 1210 through holes 1223 in the housing of the second compartment 1220. Optionally, the second compartment 1220 may comprise an ultrasound transducer 1634 at the distal end 1222 of the second compartment 1220. Optionally, the second compartment 1220 may comprise at its proximal end 1221 one or more electrical connections 1225 to exchange electrical signals with the first compartment 1210. The electrical connections 1225 may be connected to the ultrasound transducer 1634 by optional electric wires (not illustrated). The second compartment 1220 may comprise at its proximal end 1221 one or more connection means 1226A, 1226B to detachably attach the second compartment 1220 to the first compartment 1210. For example this may be a clip-on system or a system comprising magnets (e.g. a metallic piece cooperating with a magnet of the first compartment, or a magnet cooperating with a magnet or a metallic piece of the first compartment). For example, the connection means 1226A, 1226B may each comprise a recess which cooperates with a protrusion 1622A, 1622B of the first compartment 1210. (or the other way round, the recess may be in the first compartment and the protrusion may be in the second compartment). The proximal side 1221 of the second compartment 1220 with the proximal ends of the optical fibres 1611, 1612 and the connection means 1226A, 1226B may form a multifibre push-on (MPO) style connector.

Optical couplers may be provided to ensure optical communication between the proximal ends of the optical fibres 1611, 1612 of the second compartment 1220 and the distal ends of the optical fibres 1613, 1614 of the first compartment 1210. Alternatively, the tips may be butt connected.

FIGS. 9B and 9C schematically show a top view and a side view, respectively, of the hand-held device 900, with the first compartment 1210 attached to the second compartment 1220. The second compartment 1220 has been described above with reference to FIG. 9A. The first compartment 1210 may comprise one or more connection means 1622A, 1622B, forming the counterparts of the connection means 1226A, 1226B. The first compartment further comprises an optical connector 1219 that comprises the distal ends of the optical fibres 1613, 1614. The first compartment may further comprise an optical sensor 1410 optically connected to a proximal end of the one or more receiving optical fibres 1614, a light source 1490 optically connected to a proximal end of the one or more transmitting optical fibres 1613, a control unit 1420, which may comprise at least one computer processor and/or a memory. The control unit 1420 may be configured to control activation of the light source 1440, process signals received from the optical sensor 1410, and to generate the parameter based on the electrical signal generated by the optical sensor 1410.

Certain embodiments may provide optional ultrasound support. To that end, the first compartment 1210 may further comprise electrical connectors 1213 at the distal side 1212 of the first compartment 1210, corresponding to the electrical connectors 1225 at the proximal side 1221 of the second compartment 1220. The electrical connectors 1213, 1225 may be implemented by means of conductive pins and/or conductive surfaces, for example. The electrical connectors may be connected by respective electric wires to an ultrasound controller module, which may include a beamformer, for example. The ultrasound controller module 1630 may comprise circuitry for pre-processing of ultrasound signals and/or for data compression, as well as generating transmission signals for the ultrasound transducer 1634. In certain embodiments, the electric connector may alternatively be present to power and/or control any other optional electrical components in the second compartment 1220. The first compartment 1210 may further include an additional processor 1636 in communication with the processor 1420 and the ultrasound controller module 1630 for synchronization of optical measurements with ultrasound measurements.

The compartments 1210, 1220 can mechanically connect to each other at connections 1622 by any connection mechanism known in the art, such as a click mechanism or a magnetic connection. The connection mechanism and the optical fibres and the optional electric connections are aligned such that light signals travel effectively between fibres of the first compartment 1210 and the corresponding fibres of the second compartment 1220.

As indicated above, the housing 901 may comprise two detachable compartments 1210, 1220, wherein the first compartment 1210 is located at a proximal portion of the housing 901 and the second compartment 1220 is located at a distal portion of the housing 901. The second compartment 1220 comprises in its interior a plurality of optical fibres and optionally an ultrasound transducer. The transmitting optical fibre 1611, 1613 may conduct light signals to biological tissues of the examination site. The tissues may absorb, reflect, or back-scatter the light signals and generate a reflectance light signal that is collected at a distal end of the receiving fibres 1612 and guided to the receiving fibres 1614 of the first compartment 1210.

The optional ultrasound transducer 1634 may comprise, for example, acoustic lens, piezoelectric elements, backing material and electrodes, and may have electrical connections at the proximal end 1221 of the second compartment 1220.

The second component 1220 may be a low-cost component such that it is disposable, or sterilizable and reusable. In certain embodiments the second component 1220 may be useable for up to between 3 to 10 procedures. The first component 1210, on the other hand, may be more durable and may be suitable for being used for much longer time than the second component 1220.

In certain embodiments, alternatively or in addition to the optional ultrasound transducer and ultrasound control module, another imaging modality may be provided, such as optical coherence tomography.

The hand-held device 900, in particular the first compartment 1210, may further comprise a communications unit 1430. The communications unit 1430 may be configured to, under control of the control unit 1420, transmit or receive data with external devices, such as an external computer system. The communications unit 1430 may comprise any suitable communication hardware for wired or wireless transmission, such as a Wi-Fi transceiver or ethernet transceiver or universal serial bus (USB) interface. The control unit 1420 may be configured to execute commands received through the communications unit 1430. The control unit 1420 may be configured to transmit measurement data obtained from the optical sensor 1410 and/or ultrasound control module 1440 to an external device for further processing and/or display. Alternatively, the first compartment 1210 may comprise a built-in display (not illustrated) to visualize measurement results or a transducer to generate acoustic output such as alarm signals based on measurement results.

The examples and embodiments described herein serve to illustrate rather than limit the invention. The person skilled in the art will be able to design alternative embodiments without departing from the scope of the present disclosure, as defined by the appended claims and their equivalents. For example, the design of the elongated optical component may be adapted for surgery (for example as biophotonics scalpel) or for integration with other kind of probes (for example ultrasound probes).

Reference signs placed in parentheses in the claims shall not be interpreted to limit the scope of the claims. Items described as separate entities in the claims, or the description may be implemented as a single hardware item combining the features of the items described.

Certain aspects are defined in the following clauses.
1. A hand-held device (100) having a housing (200), the housing (200) comprising:
   a placeholder (222) configured to receive at least a placement member (302) of an elongated optical component (300);
   an attachment member (223) configured to removably fix the elongated optical component (300) to the housing (200), with the placement member (302) disposed in the placeholder (222);
   at least one first optical coupler (212) configured to transmit a light signal into the elongated optical component (300);
   at least one second optical coupler (213) configured to receive a light signal from the elongated optical component (300);
   an optical sensor (410), optically connected to the at least one second optical coupler (213), and configured to detect the received light signal; and
   a processor (420) configured to generate a parameter based on a signal generated by the optical sensor (410).
2. The hand-held device of clause 1, wherein
   the first optical coupler (212) is configured to transmit the light signal into an optical fibre of the elongated optical component (300), and
   the second optical coupler (213) is configured to receive the light signal from an optical fibre of the elongated optical component (300).
3. The hand-held device of any preceding clause, wherein the housing (200) comprises:
   a first compartment (210) containing the at least one first optical coupler (212), the at least one second optical coupler (213), the optical sensor (410), and the processor (420), and
   a second compartment (220) containing the placeholder (222), and the attachment member (300),
   wherein the first compartment (210) and the second compartment (220) are detachably connectable to each other.
4. The hand-held device of clause 1 or 2, wherein the housing (200) further comprises a mechanism (227) to mechanically advance at least a second elongated part of the elongated optical component (300) over a first elongated part of the elongated optical component (300) in a longitudinal direction of the elongated optical component (300), wherein the mechanism (227) is optionally spring-loaded.
5. The hand-held device of clause 4, wherein the at least one first optical coupler (212), the at least one second optical coupler (213), the optical sensor (410) and the processor (420) are comprised in a first compartment (210) of the housing (200), and wherein at least the placeholder (222), and the attachment member (300) and the mechanism (227) are comprised in a second compartment (220) of the housing (200), the first compartment (210) being detachably attachable to the second compartment (220), so that either compartment can be replaced.
6. The hand-held device of clause 5, wherein the at least one first optical coupler (212) and the at least one second optical coupler (213) are aligned with the placeholder (222) when the first compartment (210) is attached to the second compartment (220).
7. The hand-held device of any preceding clause, wherein the elongated optical component (300) is detachably attachable to the placeholder (222) by the attachment member (300), so that the elongated optical component (300) can be replaced.
8. The hand-held device of any preceding clause,
   wherein the optical sensor (410) comprises at least one spectrometer configured to detect a spectrum of the received light signal, and
   wherein the processor (420) is configured to generate the parameter based on the detected spectrum.
9. The hand-held device of any preceding clause, wherein the optical sensor (410) comprises a set of discrete detector channels (410b) and a corresponding set of photodetectors, wherein each photodetector is configured to detect light of a specific wavelength range in a detector channel of the set of discrete detector channels (410b), and wherein the processor (420) is configured to compute the parameter based on signals generated by the set of photodetectors.
10. The hand-held device of any preceding clause, wherein the housing (200) comprises an indicator configured to output information regarding the parameter.
11. The hand-held device of any preceding clause, wherein the housing (200) comprises a wireless transmitter (430) configured to wirelessly transmit the parameter to an external console.
12. The hand-held device of any preceding clause, wherein the housing (200) comprises an optical port and a light guide configured to convey optical signals received through the optical port from an external light source (440) to the at least one first optical coupler (212).
13. The hand-held device of any preceding clause, wherein the housing (200) comprises a light source (440) to generate a light signal and a light guide configured to convey the light signal generated by the light source (440) to the at least one first optical coupler (212).
14. An elongated optical component (300), comprising:
   at least two optical fibres (311, 312) extending in a longitudinal direction through the elongated optical component (300), wherein the at least two optical fibres (311, 312), are conductive to light signals, and
   a placement member (302), at a proximal end of the elongated optical component (300), comprising at least one first optical coupler (315) optically connected to at least a first one of the at least two optical fibres (311, 312) and at least one second optical coupler (316) optically connected to at least a second one of the at least two optical fibres (311, 312); and
   a connection element (303) to allow fixation of the placement member (302),
   wherein a distal end of the first one (311) of the at least two optical fibres is optically open to an exterior of the elongated optical component (300), and a distal end of the second one (312) of the at least two optical fibres is optically open to the exterior of the elongated optical component (300).
15. The elongated optical component (300) of clause 14, wherein the at least two optical fibres (311, 312) are bundled as a bundle of optical fibres within the elongated optical component (300), wherein the at least two optical fibres (311, 312) branch away from each other at a distal end (310a) of the elongated optical component (300).
16. The elongated optical component (300) of any one of clauses 14-15, wherein the elongated optical component (300) comprises a needle.
17. The elongated optical component (300) of clause 16, wherein the elongated optical component (300) comprises a biopsy needle.
18. The elongated optical component (300) of clause 16 or 17, wherein at least a distal end (310 a) of the elongated optical component (300) is hollow and comprises an insert (320), the insert (320) comprising at least one space for at least one of the optical fibres, wherein the at least one of the optical fibres (311, 312) extends through the space and is optically open to an exterior of the elongated optical component (300) at the distal end (310a) of the elongated optical component (300).
19. The elongated optical component (300) of any one of clauses 17-18, wherein:
   a distal portion of the elongated optical component (300) comprises a notch (322) adapted to accommodate a tissue sample, and
   the elongated optical component (300) further comprises:
   a cutting canula (324) slidably disposed around the elongated optical component (300); and
   a handling member (326) at a proximal end of the cutting canula (324) configured to cooperate with an external mechanism (227) to mechanically advance the cutting canula (324) over the elongated optical component (300)
20. A hand-held optical device, comprising:
   a housing (200), the housing (200) comprising:
      a placeholder (222) for receiving at least a placement member (302) of an elongated optical component (300);
      an attachment member (300) to fixate the elongated optical component (300) to the housing (200), with the placement member (302) disposed in the placeholder (222);
      at least one first optical coupler (212) configured to transmit a light signal into the elongated optical component (300);
      at least one second optical coupler (213) configured to receive a light signal from the elongated optical component (300);
      an optical sensor (410), optically connected to the at least one second optical coupler, and configured to detect the received light signal; and
      a processor (420) configured to generate a parameter based on a signal generated by the optical sensor (410),
      wherein the hand-held optical device further comprises the elongated optical component (300) comprising:
      at least two optical fibres (311, 312) extending in a longitudinal direction through the elongated optical component (300), wherein the at least two optical fibres are conductive to optical signals;
      a placement member (302) at a proximal end of the elongated optical component (300), comprising at least one first optical coupler (315) optically connected to at least one of the at least two optical fibres (311, 312); and at least one second optical coupler (316) optically connected to at least one of the at least two optical fibres (311, 312); and
      a connection element (303) to allow fixation of the placement member (302),
   wherein the housing (200) and the elongated optical component (300) comprise alignment means so that the at least one first optical coupler (315) of the elongated optical component (300) aligned with said at least one first optical coupler (212) of the housing and the at least one second optical coupler (316) of the elongated optical component (300) is aligned with said at least one second optical coupler (213) of the housing, and the placement member (302) is removably fixable the placeholder (222) by the connection element and the attachment member (300).
21. The device of any preceding clause, wherein the elongated optical component (300) is disposable.
22. A method to assemble a biophotonics device, the method comprising:
   providing the hand-held device according to any one of clauses 1-13;
   providing the elongated optical component (300) according to any one of clauses 14-19;
   inserting at least the placement member (302) of the elongated optical component (300) in the placeholder (222) of the housing (200); and
   connecting the attachment member (300) of the hand-held device, to the connection element of the elongated optical component (300).
23. A hand-held optical device, comprising a housing (600), the housing (600) comprising:
   at least one transmitting optical fibre (311) and at least one receiving optical fibre (312);
   an optical sensor (410), optically connected to the at least one receiving optical fibre (312) and configured to detect a light signal;
   a light source (440) optically connected to the at least one transmitting optical fibre (311) and configured to generate a light signal; and
   a processor (420) configured to control activation of the light source (440) and to generate a parameter based on a signal generated by the optical sensor (410),
   wherein a distal end of the transmitting optical fibre (311) is configured to optically communicate with an outside of the housing (600) and a distal end of the receiving optical fibre (312) is configured to optically communicate with an outside of the housing (600).

1. A hand-held device having a housing, the housing comprising a first compartment and a second compartment,
wherein the first compartment contains: at least one first optical coupler configured to transmit a light signal into an elongated optical component, at least one second optical coupler configured to receive a light signal from the elongated optical component an optical sensor optically connected to the at least one second optical coupler and configured to detect the received light signal, and a processor configured to generate a parameter based on a signal generated by the optical sensor;
wherein the second compartment contains:
a placeholder configured to receive at least a placement member of the elongated optical component and
an attachment member configured to removably fix the elongated optical component to the housing, with the placement member disposed in the placeholder; and
wherein the first compartment and the second compartment are detachably attachable to each other.

2. The hand-held device of claim 1, wherein
the first optical coupler is configured to transmit the light signal into an optical fibre of the elongated optical component and
the second optical coupler is configured to receive the light signal from an optical fibre of the elongated optical component.

3. The hand-held device of claim 1, wherein the second compartment further comprises a mechanism to mechanically advance at least a second elongated part of the elongated optical component over a first elongated part of the elongated optical component in a longitudinal direction of the elongated optical component, wherein the mechanism is optionally spring-loaded.

4. The hand-held device of claim 1, wherein the at least one first optical coupler and the at least one second optical coupler are aligned with the placeholder when the first compartment is attached to the second compartment.

5. The hand-held device of claim 1, wherein the elongated optical component is detachably attachable to the placeholder by the attachment member, so that the elongated optical component can be replaced.

6. The hand-held device of claim 1,
wherein the optical sensor comprises at least one spectrometer configured to detect a spectrum of the received light signal, and
wherein the processor is configured to generate the parameter based on the detected spectrum.

7. The hand-held device of claim 1, wherein the optical sensor comprises a set of discrete detector channels and a corresponding set of photodetectors, wherein each photodetector is configured to detect light of a specific wavelength range in a detector channel of the set of discrete detector channels, and wherein the processor is configured to compute the parameter based on signals generated by the set of photodetectors.

8. The hand-held device of claim 1, wherein the housing comprises an indicator configured to output information regarding the parameter.

9. The hand-held device of claim 1, wherein the housing comprises a wireless transmitter configured to wirelessly transmit the parameter to an external console.

10. The hand-held device of claim 1, wherein the housing comprises an optical port and a light guide configured to convey optical signals received through the optical port from an external light source to the at least one first optical coupler.

11. The hand-held device of claim 1, wherein the housing comprises a light source to generate a light signal and a light guide configured to convey the light signal generated by the light source to the at least one first optical coupler.

12. An elongated optical component for the hand-held device of claim 1, comprising:
at least two optical fibres extending in a longitudinal direction through the elongated optical component, wherein the at least two optical fibres, are conductive to light signals, and
a placement member, at a proximal end of the elongated optical component comprising at least one first optical coupler optically connected to at least a first one of the at least two optical fibres and at least one second optical coupler optically connected to at least a second one of the at least two optical fibres; and
a connection element to allow fixation of the placement member,
wherein a distal end of the first one of the at least two optical fibres is optically open to an exterior of the elongated optical component, and a distal end of the second one of the at least two optical fibres is optically open to the exterior of the elongated optical component.

13. The elongated optical component of claim 12, wherein the at least two optical fibres are bundled as a bundle of optical fibres within the elongated optical component, wherein the at least two optical fibres branch away from each other at a distal end of the elongated optical component.

14. The elongated optical component of claim 12, wherein the elongated optical component comprises a needle.

15. The elongated optical component of claim 14, wherein the elongated optical component comprises a biopsy needle.

16. The elongated optical component of claim 14, wherein at least a distal end of the elongated optical component is hollow and comprises an insert, the insert comprising at least one space for at least one of the optical fibres, wherein the at least one of the optical fibres extends through the space and is optically open to an exterior of the elongated optical component at the distal end of the elongated optical component.

17. The elongated optical component of claim 16, wherein:
a distal portion of the elongated optical component comprises a notch adapted to accommodate a tissue sample, and
the elongated optical component further comprises:
a cutting canula slidably disposed around the elongated optical component; and
a handling member at a proximal end of the cutting canula configured to cooperate with an external mechanism to mechanically advance the cutting canula over the elongated optical component.

18. The hand-held optical device according to claim 1, comprising:
wherein the hand-held optical device further comprises the elongated optical component comprising:
at least two optical fibres extending in a longitudinal direction through the elongated optical component, wherein the at least two optical fibres are conductive to optical signals;
a placement member at a proximal end of the elongated optical component, comprising at least one first optical coupler optically connected to at least one of the at least two optical fibres; and at least one second optical coupler (316) optically connected to at least one of the at least two optical fibres; and
a connection element to allow fixation of the placement member,
wherein the housing and the elongated optical component comprise alignment means so that the at least one first optical coupler of the elongated optical component is aligned with said at least one first optical coupler of the housing and the at least one second optical coupler of the elongated optical component is aligned with said at least one second optical coupler of the housing, and the placement member is removably fixable in the placeholder by the connection element and the attachment member.

19. The device of claim 1, wherein the elongated optical component is disposable.

20. A method to assemble a biophotonics device, the method comprising:
providing the hand-held device according to claim 1;
providing an elongated optical component comprising:
at least two optical fibres extending in a longitudinal direction through the elongated optical component, wherein the at least two optical fibres, are conductive to light signals, and
a placement member, at a proximal end of the elongated optical component, comprising at least one first optical coupler optically connected to at least a first one of the at least two optical fibres and at least one second optical coupler optically connected to at least a second one of the at least two optical fibres; and
a connection element to allow fixation of the placement member,
wherein a distal end of the first one of the at least two optical fibres is optically open to an exterior of the elongated optical component, and a distal end of the second one of the at least two optical fibres is optically open to the exterior of the elongated optical component;
inserting at least the placement member of the elongated optical component in the placeholder of the second compartment of the housing; and
connecting the attachment member of the second compartment of the housing to the connection element of the elongated optical component

21. A hand-held optical device, comprising a housing, the housing comprising:
a first compartment comprising:
at least one transmitting optical fibre extending inside the first compartment from a distal side of the first compartment,
at least one receiving optical fibre extending inside the first compartment from the distal side of the first compartment
an optical sensor optically connected to the at least one receiving optical fibre and configured to detect a light signal,
a light source optically connected to the at least one transmitting optical fibre and configured to generate a light signal,
a processor configured to control activation of the light source and to generate a parameter based on a signal generated by the optical sensor and connection means;
the housing further comprising a second compartment comprising:
at least one transmitting optical fibre extending from a proximal side of the second compartment to a distal side of the second compartment
at least one receiving optical fibre extending from the proximal side of the second compartment to a distal side of the second compartment, and
connection means configured to cooperate with the connection means of the first compartment to detachably attach the first compartment to the second compartment,
wherein a distal end of the transmitting optical fibre of the second compartment is configured to optically communicate with an outside of the housing and a distal end of the receiving optical fibre of the second compartment is configured to optically communicate with an outside of the housing, and
wherein the connection means of the first compartment and the distal end of the transmitting optical fibre of the first compartment and the distal end of the receiving optical fibre of the first compartment are aligned with the corresponding connection means of the second compartment and the corresponding proximal end of the transmitting optical fibre of the second compartment and the corresponding proximal end of the receiving optical fibre of the second compartment so that the optical fibres of the first compartment are in optical communication with the optical fibres of the second compartment when the first compartment is attached to the second compartment by the connection means of both compartments.

22. The hand-held optical device according to claim 21, wherein
the first compartment comprises at least one electrical connection at the distal side of the first compartment; and
the second compartment comprises at least one electrical connection at the proximal side of the second compartment;
wherein the at least one electrical connection at the distal side of the first compartment is aligned with the at least one electrical connection at the proximal side of the second compartment to allow transmission of electrical signals when the first compartment is attached to the second compartment by the connection means of both compartments.
